(19) United States
(12) Patent Application Publication
(10) Pub. No.: US 2009/0076411 A1
(43) Pub. Date: Mar. 19, 2009

(54) Endometrial anesthesia delivery device and methods of use thereof

(76) Inventor: Syed Rizvi, Hemet, CA (US)

Correspondence Address: Syed Rizvi, #112, 200 North Yale Street, Hemet, CA, 92544, US

(21) Appl. No.: 11/901,409

(22) Filed: Sep. 17, 2007

(51) Int. Cl. A61B 10/00; A61B 17/42; A61B 5/103

(52) U.S. Cl. 600/562; 600/587; 606/119

(57) ABSTRACT

The field of the present invention relates to medical devices. More particularly, the field of the present invention relates to devices for providing anesthesia to an endometrial cavity of a patient and, furthermore, to methods of using such devices for endometrial biopsy and other medical procedures. According to certain preferred embodiments of the invention, devices for delivering anesthesia to an endometrial cavity are provided, which generally comprise (i) a primary shaft, (ii) a port located in a backside portion of the device, and (iii) a substantially spherical tip.

### FIELD OF THE INVENTION

The field of the present invention relates to medical devices. More particularly, the field of the present invention relates to devices for sounding and/or providing anesthesia to an endometrial cavity of a patient and, furthermore, to methods of using such devices for endometrial biopsy and other medical procedures.

### BACKGROUND OF THE INVENTION

An endometrial biopsy is a procedure used by physicians to obtain a small sample of the lining of a patient's uterus (endometrium), which is subsequently examined via microscopy.

There are several reasons for performing an endometrial biopsy on a patient. For example, when a woman is having a difficult time becoming pregnant, an endometrial biopsy is often performed to determine whether the lining of her uterus can support a pregnancy. An endometrial biopsy may also be carried out to locate the cause of abnormal uterine bleeding, to check for overgrowth of the lining (endometrial hyperplasia), or to check for cancer.

There are several methods used by physicians to perform an endometrial biopsy. For example, a straw-like device may be employed to suction a small sample of lining from the uterus. Another method involves the use of an electronic suction device (Vabra aspiration). This method is often uncomfortable and, at times, painful to the patient. Still further, a sharp-edged tool known as a curette is often used to scrape a small tissue sample (and collect the tissue with a syringe or suction). This technique is often referred to as a “dilation and curettage” procedure (D&C). A D&C procedure may be performed to control heavy uterine bleeding (hemorrhage) or to help identify the cause of uterine bleeding. In most cases, such D&C procedures are performed with general or, more preferably, regional anesthesia.

Various medical devices have been developed over the years, which may be used by physicians to perform endometrial biopsies. Many of these devices, however, are deficient in one or more ways. More particularly, many of the currently-available (or prior art) devices that are used to deploy regional anesthesia prior to endometrial biopsy procedures, e.g., D&C procedures, are integrally formed with a device that is also used for cutting and/or harvesting the tissue. Such “bifunctional” instruments are often cumbersome, difficult to maneuver, and more expensive to manufacture. Furthermore, such “bifunctional” instruments are not amenable to manufacturing single-use, disposable devices, insofar as such devices are more expensive to manufacture. In addition, many of the currently-available anesthesia delivery devices are unable to effectively deploy anesthesia over the target area in an efficient, thorough, safe, and comfortable manner.

In addition, other methods currently employed by surgeons involve the use of a rudimentary syringe (or similar devices). Indeed, in some cases, surgeons have been known to press a syringe against the opening of a patient's cervix and deploying anesthetic thereon. Needless to say, such techniques are not preferred, insofar as the anesthetic often does not reach the endometrial cavity (and, in large part, remains in the vaginal area) leading to insufficient anesthesia. This problem is particularly relevant in patients with stenotic cervical os, unusual anatomy, or abnormalities (such as fibroids).

In view of the foregoing, there is a continuing need for improved anesthesia delivery devices and methods of use thereof.

### SUMMARY OF THE INVENTION

According to a first preferred embodiment of the present invention, a device is provided for delivering anesthesia to an endometrial cavity of a patient. The device generally comprises (i) a primary shaft, (ii) a port located in the backside portion of the device, and (iii) a substantially spherical tip. In certain embodiments, the primary shaft comprises a curved configuration, which provides a displacement between the tip and backside portion of the device ranging between about 0.5 and 1.5 cm. The port of the device preferably comprises an axis that is substantially parallel and contiguous with the axis of the primary shaft.

According to another preferred embodiment of the present invention, a uterine sound is provided, which may be used by a surgeon to sound a patient's uterine cavity and deliver local or regional anesthesia to such cavity.

According to additional preferred embodiments of the present invention, methods for anesthetizing the endometrial cavity of a patient, along with methods of performing an endometrial biopsy, are provided. Such methods generally comprise providing anesthesia to an endometrial cavity of a patient using a device described herein. Such methods may further comprise extracting a sample of endometrial tissue using an instrument other than the anesthesia delivery device.

The above-mentioned and additional features of the present invention are further illustrated in the Detailed Description contained herein.

### BRIEF DESCRIPTION OF THE DRAWINGS

This patent application includes certain drawings executed in color. A copy of this application, with color drawings, may be obtained from the U.S. Patent Office, upon submitting an appropriate request and the requisite fee.

FIG. 1 is a top, side and ¾ view of a device of the present invention.

FIG. 2 is a top and side view of a device of the present invention.

FIG. 3 is a side view of a device of the present invention.

FIG. 4 is a side, front, top and ¾ view of different tip configurations that may be used in the device of the present invention.

FIG. 5 is a diagram showing certain steps of using the device of the present invention, namely, attaching a syringe to the device.

FIG. 6 is a diagram showing certain steps of using the device of the present invention, namely, inserting the device into the endometrial cavity of a patient while continuously injecting anesthetic therein.

FIG. 7 is a diagram showing certain steps of using the device of the present invention, namely, using the device to sound the patient's uterus and inject the remaining anesthetic into the endometrial cavity of the patient.

FIG. 8 is a diagram showing certain steps of using the device of the present invention, namely, withdrawing the device of the present invention from the endometrial cavity of the patient.

### DETAILED DESCRIPTION OF THE INVENTION

The following will describe in detail several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

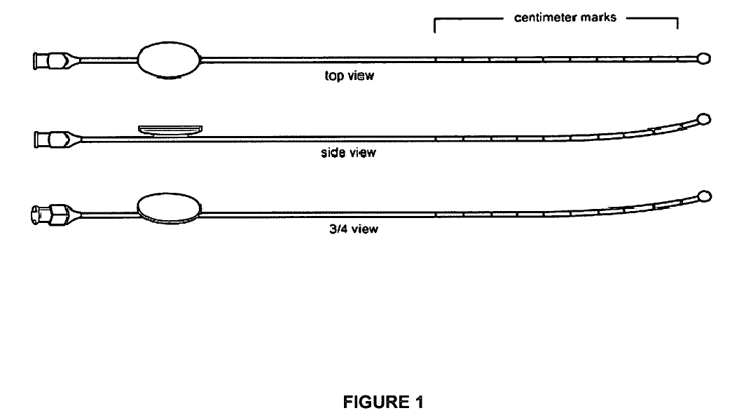

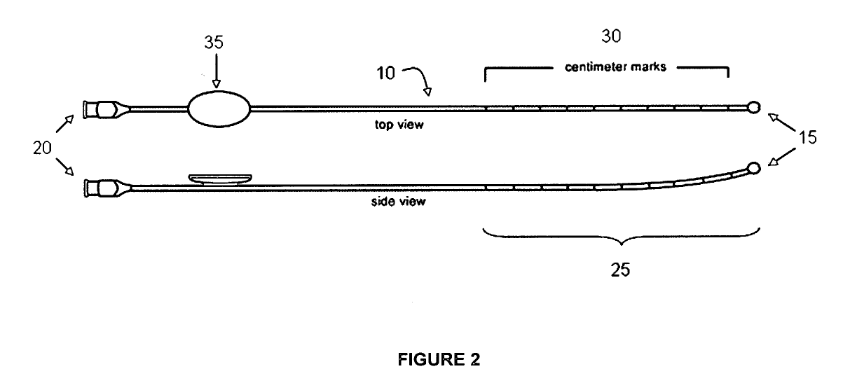

According to a first preferred embodiment of the present invention, as generally shown in FIG. 1, a device is provided for delivering anesthesia to an endometrial cavity of a patient. More particularly, referring to FIG. 2, the device generally comprises (i) a primary shaft 10, (ii) a port 20 located in the backside portion of the device, and (iii) a substantially spherical tip 15. The primary shaft 10 is preferably constructed to enclose a volume and make exhibit any of various configurations. The primary shaft 10 will preferably exhibit a cylindrical, or substantially cylindrical, shape. The primary shaft 10, however, may exhibit other geometric configurations which are capable of being safely and relatively comfortably inserted into the vaginal and endocervical canal as shown and described herein.

The device of the present invention may optionally include a thumb rest 35, which supports a surgeon's thumb during use of the device. In addition, referring to FIG. 2, the primary shaft 10 may optionally be provided with distance marks, such as the centimeter marks 30 shown in FIG. 2, which may be used to monitor and measure the distance of insertion into a patient (as well as measure the length of a patient's uterus and endometrial cavity).

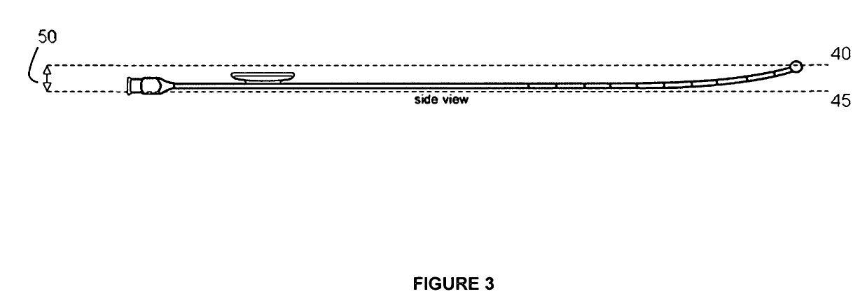

In certain preferred embodiments of the present invention, referring to FIG. 2, the primary shaft 10 comprises a curved configuration 25, which provides a displacement between the tip and backside portion of the device. More particularly, referring to FIGS. 2 and 3, the backside portion of the device 45 generally refers to a line (or plane) that is tangential to the portion of the primary shaft 10 that is closest to the device port 20. The displacement between the tip and backside portion of the device is therefore the distance 50 between such tangential line (or plane) 45 and a line (or plane) 40 that is tangential to a portion of the tip 15 that is most distant from the tangential line (or plane) 45 that is tangential to the above-referenced portion of the primary shaft 10.

According to such embodiments, referring to FIG. 3, the curved configuration provides a displacement 50 that preferably ranges between about 0.5 and 1.5 cm, or more preferably between about 0.75 and 1.25 cm, or still more preferably between about 0.90 and 1.10 cm. According to such embodiments, the length of the primary shaft 10, beginning at about the thumb rest 35 to the tip 15, may range between about 15 and 25 cm, such as about 18-20 cm. The invention provides that the curved configuration 25 providing the displacement 50 described above has been shown to be particularly well-suited and adapted to the natural shape of the endocervical canal and endometrial cavity of most women. That is, the invention provides that the curved configuration 25 providing the displacement 50 described above is specially designed to match the contours of a woman's endometrial cavity, thereby facilitating safe and more comfortable insertion therein (and more accurate dispersion of anesthesia).

The port 20 of the device is adapted to receive the anesthesia. For example, the port 20 may comprise a pore that is adapted to receive a syringe (either with or without a needle) that is loaded with anesthesia. Upon deploying the anesthesia from the syringe into the port 20 (e.g., by inserting the syringe into a pore located in the port 20), the anesthesia is provided to and forced through the interior cavity of the primary shaft 10 until it reaches the tip 15. Upon reaching the tip 15, the anesthesia is forced into the endometrial cavity of a patient through one or more pores located in the tip 15.

In certain preferred embodiments, the port 20 is located in the backside portion of the device, such that an axis through the port 20 is substantially parallel and contiguous with the axis of the primary shaft 10. More particularly, the invention provides that the port 20 is located in the backside portion of the device, such that when anesthesia is deployed therein is travels directly down and through the interior cavity of the primary shaft 10. The invention provides that locating the port 20 in the backside portion of the device enables a surgeon to deploy the anesthesia therein with a preferred amount of force, which is proportional to the force with which the anesthesia is subsequently dispersed from the tip 15. When anesthesia is subsequently dispersed from the tip 15 with a greater amount of force, the anesthesia is more widely and thoroughly deployed therefrom (which enables a greater surface area of an endometrial cavity to be contacted with the anesthesia). The invention provides that certain prior art devices are designed such that a port is located on the side of a shaft, which is adapted to receive anesthesia. Such a design is not preferred, insofar as anesthesia injected through such port will first contact an interior side wall of a primary shaft, thereby reducing the force with which such anesthesia is deployed from the tip portion thereof.

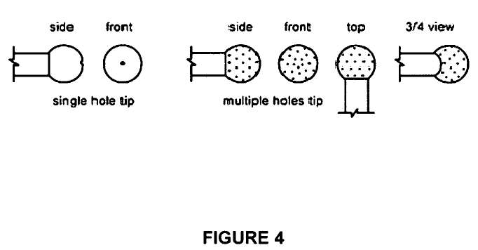

As used herein, the term “substantially spherical tip” includes the ball-like tip that is located on the distal portion of the primary shaft 10, as shown in FIGS. 1 and 2, but further includes any other shape that does not exhibit an edge (or other protrusion) that could lacerate or puncture the vaginal canal, endocervical canal, or endometrial cavity of a patient. Referring to FIG. 4, the tip 15 may comprise a single pore through which anesthesia may be dispensed. Alternatively, the tip 15 may comprise a plurality of pores, which may serve to more quickly disperse anesthesia into various parts of an endometrial cavity.

In certain preferred embodiments, the pore(s) that are located in the tip 15 are configured to dispense the anesthesia in the form of a spray or mist. In other words, in such embodiments, the pores are not merely apertures located in the tip 15, but rather the pores are designed to emit the anesthesia in a spray- or mist-like fashion (similar to the way the nozzle on a household cleaner bottle is often adapted to dispense cleaner in the form of a spray or mist). Of course, in such embodiments, by dispensing the anesthesia in the form of a spray or mist, a greater surface area of a patient's endometrial cavity will be efficiently and quickly contacted with the anesthetic, resulting in a more thorough and quick anesthesia.

The anesthesia delivery device of the present invention may be constructed of any suitable material. For example, the device may be constructed of stainless steel, plastics, elastomers, any suitable polymer, or combinations thereof. In addition, the invention provides that the device may be used to deliver anesthesia to the endometrial cavity of a patient, sterilized, and re-used with another patient. Alternatively, the device of the present invention may be constructed as a single-use, disposable instrument.

According to another preferred embodiment of the present invention, methods for performing an endometrial biopsy on a patient are provided. Such methods generally comprise providing anesthesia to an endometrial cavity of a patient using a device described herein. The methods further comprise extracting a section of endometrial tissue using an instrument other than the anesthesia delivery device.

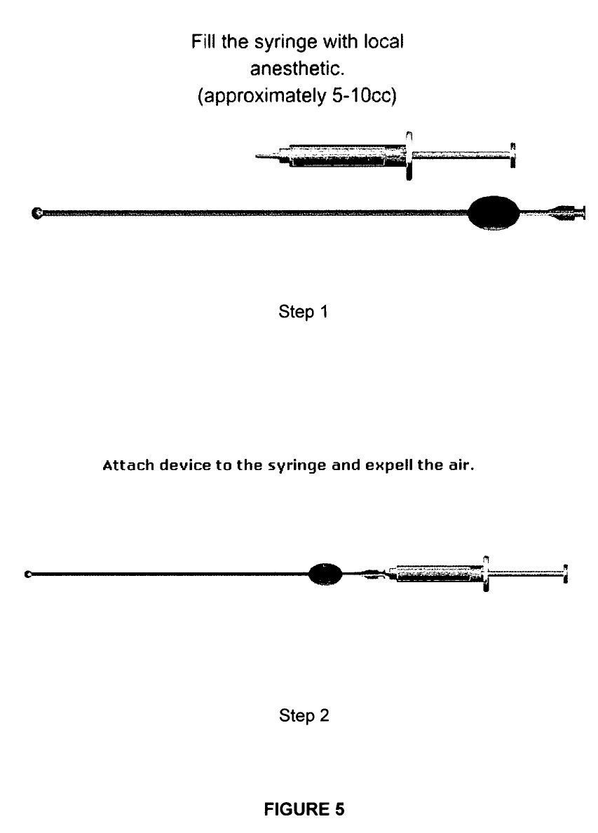

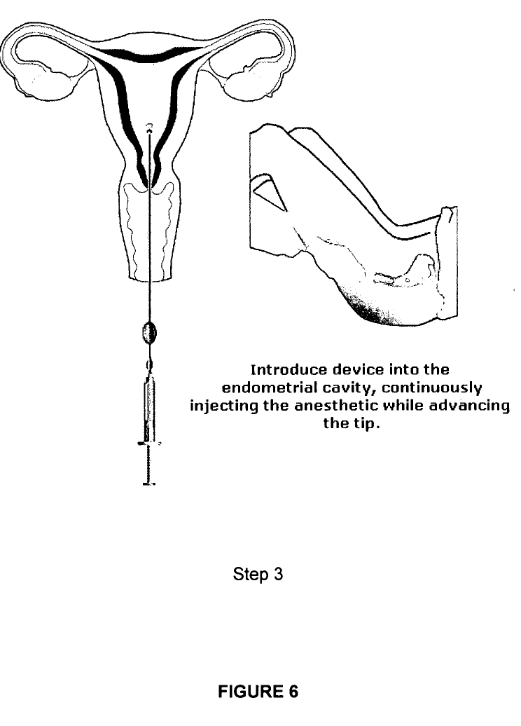

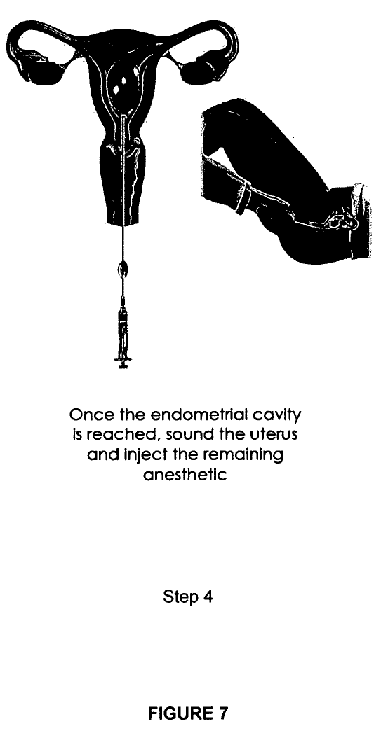

More particularly, referring to FIG. 5, a syringe may be filled with local anesthetic, such as approximately 5-10 mLs (CCs) of anesthetic. Next, the syringe is attached (or operably engaged) with the port 20 of the device—either with or without the use of a needle connected to the syringe. Next, referring to FIG. 6, the tip 15 (along with a portion of the primary shaft 10) of the device is gently inserted into the endometrial cavity of the patient. As the tip 15 is introduced into the endometrial cavity, a surgeon preferably continuously injects (or dispenses) anesthetic into the patient's endometrial cavity. Referring to FIG. 6, for example, a plunger connected to the syringe is pushed to inject anesthetic into the port 20, down the interior portion of the primary shaft 10, and out through the pore(s) in the tip 15—into the patient's endometrial cavity. Referring to FIG. 7, once the endometrial cavity has been reached, the device should be used to sound the patient's uterus and to dispense the remaining anesthetic therein. As used herein, “sounding the patient's uterus,” “sounding the patient's uterine cavity,” and like phrases, refer to a procedure whereby the device described herein is used by a surgeon to measure the depth and course of a patient's uterus (or uterine cavity). As mentioned above, the distance markings 30 on the primary shaft 10 make the device particularly well-suited for sounding the uterine cavity of a patient.

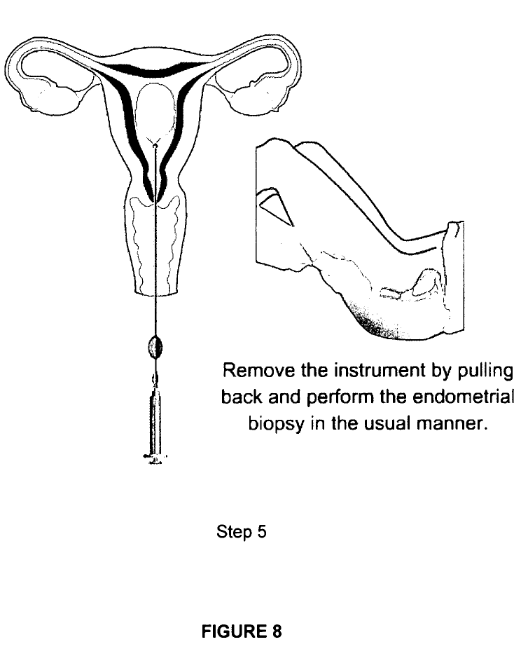

Referring to FIG. 8, once the anesthetic has been dispensed, the device is pulled backwards, out of the patient's endometrial cavity. Next, an endometrial biopsy is performed using a desired procedure and instrument of choice. Importantly, however, the invention provides that the endometrial biopsy is preferably performed using an instrument other than the anesthesia delivery device described herein. The invention provides that using instruments that are dedicated to delivering anesthesia, or cutting and harvesting endometrial tissue, eliminates the need for cumbersome medical devices that purport to be capable of both functions. In reality, such bifunctional devices are difficult to manage, use, and clean.

Because a surgeon may also use the device described herein to sound a patient's uterine cavity (as mentioned above), the invention provides that another preferred embodiment is a novel uterine sound, which may be used by a surgeon to safely and comfortably sound a patient's uterine cavity and deliver local or regional anesthesia to such cavity.

The anesthesia delivery devices (and uterine sounds) of the present invention, and methods of use thereof, provide many significant advantages over the devices and methods of the prior art. For example, since the anesthesia delivery device of the present invention is dedicated to the delivery of anesthesia (and does not serve multiple functions), a surgeon is not restricted in the type of biopsy instrument and/or procedure that he or she wishes to employ.

The tip 15 of the device is specially designed (a) to reduce the risk of uterine perforation, (b) to more easily glide through the endocervical canal (which is particularly important for use with stenotic endocervical canals, i.e., in post-menopausal women, or in navigating around obstructions in the endocervical canal, i.e., around fibroids obstructing the endocervical canal or other anatomical abnormalities in the uterus), and (c) to enable rapid and widespread application of anesthetic to the lining of an endometrial cavity (resulting in faster and more effective analgesia).

The curved configuration 25 of the primary shaft 10 also provides many distinct advantages over the prior art. In particular, for example, such design (a) conforms to the natural curve of the uterine cavity, allowing for easy and more comfortable insertion and (b) allows for the determination of the shape of a patient's uterus (e.g., an anteverted uterus versus a retroverted uterus), which decreases the risk of uterine perforation during subsequent procedures (and is also important in evaluating a patient's uterus for certain conditions).

Still further, the markings 30 on the primary shaft 10 provide a number of advantages, namely, to safely measure the length of the endometrial cavity. Such a determination is important in evaluating a patient's uterus for any of various health conditions and, furthermore, to prevent uterine perforation during subsequent procedures—using other devices.

It is to be understood that both the general description and the detailed description are exemplary and explanatory, but are not restrictive of the invention. The invention in its broader aspects is not limited to the specific details shown and described, and departures may be made from such details without departing from the principles of the invention and without sacrificing its chief advantages.

What is claimed is:

1. A device for delivering anesthesia to an endometrial cavity, which comprises:
(a) a primary shaft,
(b) a port located in a backside portion of the device, and
(c) a substantially spherical tip.

2. The device of claim 1, wherein the primary shaft comprises a curved configuration.

3. The device of claim 2, wherein the curved configuration provides a displacement between the tip and backside portion of the device ranging between 0.5 and 1.5 cm.

4. The device of claim 3, wherein the displacement ranges between 0.75 and 1.25 cm.

5. The device of claim 4, wherein the curved configuration accommodates the shape and contour of the endometrial cavity.

6. The device of claim 1, wherein the tip is configured to spray anesthesia into the endometrial cavity.

7. The device of claim 6, wherein the tip comprises a single pore through which anesthesia is sprayed.

8. The device of claim 6, wherein the tip comprises a plurality of pores through which anesthesia is sprayed.

9. The device of claim 6, wherein the port comprises an axis that is substantially parallel and contiguous with an axis of the primary shaft.

10. The device of claim 9, which further comprises a thumb pad.

11. The device of claim 10, wherein the primary shaft comprises distance markers.

12. A device for delivering anesthesia to an endometrial cavity, which comprises:
(a) a substantially spherical tip that is configured to spray anesthesia into the endometrial cavity;
(b) a primary shaft, wherein the primary shaft comprises a curved configuration that provides a displacement between the tip and backside portion of the device ranging between 0.75 to 1.25 cm; and
(c) a port located in a backside portion of the device, wherein the port comprises an axis that is substantially parallel and contiguous with an axis of the primary shaft.

13. A method for performing an endometrial biopsy, which comprises:
(a) providing anesthesia to an endometrial cavity using an anesthesia delivery device, wherein said device comprises:
(i) a primary shaft,
(ii) a port located in a backside portion of the device, and
(iii) a substantially spherical tip; and
(b) extracting a section of endometrial tissue using an instrument other than said anesthesia delivery device.

14. The method of claim 13, wherein the primary shaft of the anesthesia delivery device comprises a curved configuration.

15. The method of claim 14, wherein the curved configuration provides a displacement between the tip and backside portion of the anesthesia delivery device ranging between 0.75 to 1.25 cm.

16. The method of claim 15, wherein the curved configuration accommodates the shape and contour of the endometrial cavity.

17. The method of claim 13, wherein the tip is configured to spray anesthesia into the endometrial cavity.

18. The method of claim 13, further comprising the step of using the device to sound the patient's uterine cavity prior to extracting a section of the endometrial tissue.

19. The method of claim 13, wherein the tip of the anesthesia delivery device comprises (i) a single pore or (ii) a plurality of pores through which anesthesia is sprayed.

20. The method of claim 13, wherein the port of the anesthesia delivery device comprises an axis that is substantially parallel and contiguous with an axis of the primary shaft.
